(19) United States
(12) Patent Application Publication
(10) Pub. No.: US 2014/0203555 A1
(43) Pub. Date: Jul. 24, 2014

(54) PROXIMAL FITTING FOR A CATHETER

(71) Applicant: Cook Medical Technologies LLC, Bloomington, IN (US)

(72) Inventors: Gregory A. Frankland, Bloomington, IN (US); Karol Lagodzki, Bloomington, IN (US); Tyler P. Turk, Greenwood, IN (US)

(73) Assignee: Cook Medical Technologies LLC, Bloomington, IN (US)

(21) Appl. No.: 14/162,128

(22) Filed: Jan. 23, 2014

Related U.S. Application Data
(60) Provisional application No. 61/756,207, filed on Jan. 24, 2013.

(51) Int. Cl. A61M 25/01

(52) U.S. Cl. CPC A61M 25/0172; A61M 25/0169
USPC 285/390

(57) ABSTRACT

A proximal fitting for a catheter is provided for manipulating a guide wire. The proximal fitting includes a first hub and a second hub that engage one another. A collet having a passage can be inserted into the passage of the first hub while being unable to pass through the passages of the first and second hubs. A first portion of the collet has a convex angular surface and a slot that extends radially from the passage in at least one direction. The convex angular surface of the collet interfaces with a concave angular surface on the first hub or the second hub thereby compressing the passage of the collet in response to the first and second hubs engaging one another.

This application claims priority to U.S. Provisional Application No. 61/756,207, filed Jan. 24, 2013, which is hereby incorporated by reference herein.

### BACKGROUND

The present invention relates generally to medical devices and more particularly to a proximal fitting for a catheter.

Minimally invasive medical procedures have become common in the medical profession due to the lower risk and trauma associated with minimally invasive procedures and the lower cost compared to open surgical procedures. Minimally invasive procedures generally involve gaining access to a patient's internal vessel by puncturing the patient's skin, intermediate tissues between the skin and the vessel, and the wall of the vessel. An elongate medical instrument may then be inserted through the access site so that the distal end of the medical instrument is located within the patient's internal vessel, while the proximal end of the medical instrument remains outside the patient's body. The physician may then manipulate the proximal end of the medical instrument outside the patient's body to move and orient the distal end of the medical instrument to a location within the vessel where treatment is needed. Thus, a treatment site within a patient's vessel may be treated from outside the patient's body through a relatively small access site that is located some distance from the treatment site. By contrast, conventional open surgical procedures would require opening the tissues immediately adjacent the treatment site so that the surgeon can gain direct access to the treatment site.

During many common minimally invasive medical procedures, a catheter and a guide wire are used in combination. The distal end of the guide wire is inserted into the patient's vessel though the access site. A torque device that can help grip, hold and manipulate the guide wire is often attached to a proximal end of the guide wire to help a physician control movement of the relatively thin guide wire. With the help of the torque device, the physician can move the distal end of the guide wire to the treatment site. The torque device is then removed to be able to slide the distal end of the catheter over the proximal end of the guide wire. The distal end of the catheter can then be moved to the treatment site over the guide wire. Typically, the guide wire is removed from the catheter and the vessel after the catheter reaches the target site. However, after inserting the catheter, the position of the catheter and guide wire may need to be adjusted. The torque device can be re-attached to the proximal end of the guide wire to assist the physician again with manipulating the proximal end to move and orient the distal end of the guide wire. Therefore, having to attach and detach the torque device can be time-consuming. Furthermore, many torque devices are not simple to attach to the guide wire and attachment may require a complicated procedure. Therefore, the inventors believe an improved device to manipulate the guide wire would be desirable.

### SUMMARY

A proximal fitting for a catheter is described for manipulating a guide wire. The proximal fitting includes a first hub having a passage extending from a first end to a second end of the first hub, and the first hub has first threads. The proximal fitting also includes a second hub having a passage extending from a first end to a second end of the second hub, and the second hub has second threads that mate with the first threads of the first hub. The first end of the first hub and the second end of the second hub being disposed toward each other, and the second end of the first hub and the first end of the second hub being disposed away from each other. The proximal fitting further has a collet having a passage extending from a first end to a second end of the collet. The collet is configured to be inserted into the first end of the passage of the first hub while being unable to pass through the second end of the passage of the first hub and being unable to pass through the first end of the passage of the second hub. A first portion of the collet extends from the first end to a position between the first end and the second end, and a second portion of the collet extends from the second end to the position. The first portion of the collet has a convex angular surface and at least one slot that extends radially from the passage in at least one direction. The convex angular surface of the collet interfaces with a concave angular surface on the first hub or the second hub thereby compressing the passage of the collet in response to the first and second threads engaging one another.

### BRIEF DESCRIPTION OF SEVERAL VIEWS OF THE DRAWINGS

The invention may be more fully understood by reading the following description in conjunction with the drawings, in which:

FIG. 1 is a perspective view of a proximal fitting coupled to a catheter with a guide wire positioned within the proximal fitting and catheter;

FIG. 2 is an exploded perspective view of the proximal fitting, catheter, and guide wire of FIG. 1 showing a first hub, a second hub, and a collet of the proximal fitting;

FIG. 3 is a cross-sectional view through an axis of the proximal fitting, catheter, and guide wire of FIG. 1;

FIG. 4 is a head on view of a first end of the collet of FIG. 2 with the guide wire positioned within the passage of the collet; and

FIG. 5 is a perspective view of the second hub of FIG. 2.

### DETAILED DESCRIPTION

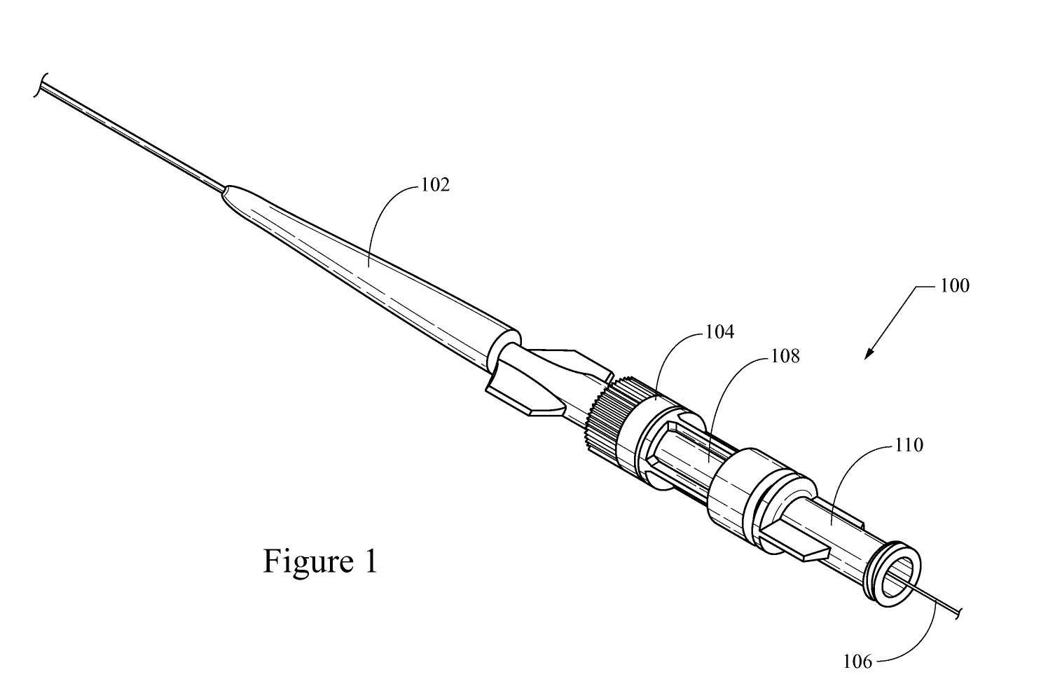

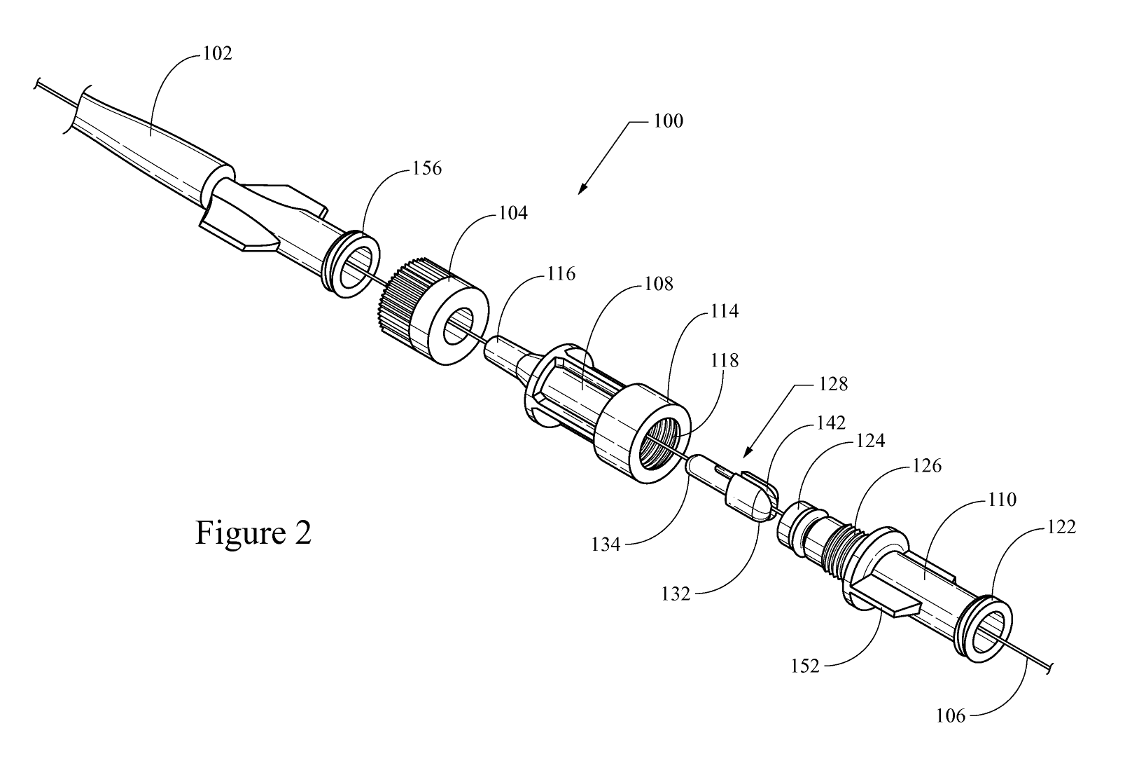

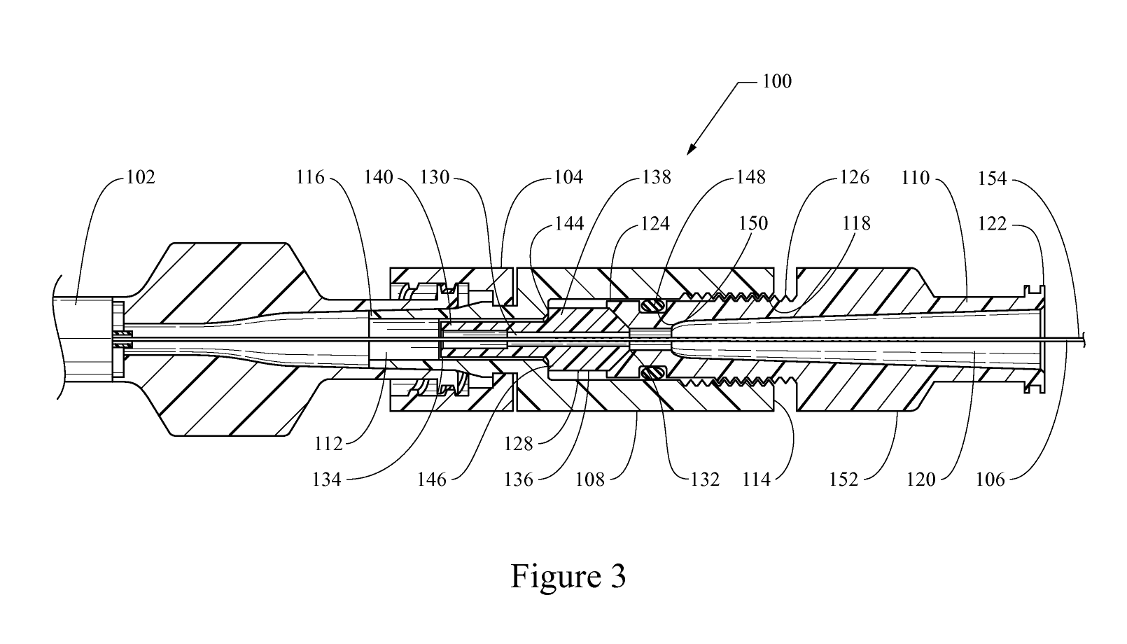

Referring now to the figures, and particularly to FIG. 1, a proximal fitting 100 coupled to a catheter 102 by a snap cap 104 is shown. A guide wire 106 can be inserted through the proximal fitting 100 and the catheter 102. The proximal fitting 100 includes a first hub 108 and a second hub 110. To further show the components of the proximal fitting 100, FIG. 2 is an exploded view of the proximal fitting 100 of FIG. 1 and FIG. 3 is a longitudinal cross-section through an axis of the proximal fitting 100.

The first hub 108 has a passage 112 that extends from a first end 114 to a second end 116 of the first hub 108. The first end 114 can be a proximal end and the second end 116 can be a distal end. The first hub 108 can have first threads 118. The first end 114 of the first hub 108 can have the first threads 118 such as internal threads on an internal surface of the first hub 108 such as within the passage 112. However, the first threads 118 can instead be external threads on an outer surface of the first hub 108.

The second hub 110 has a passage 120 that extends from a first end 122 to a second end 124 of the second hub 110. The first end 122 can be a proximal end and the second end 124 can be a distal end. The second hub 110 can have second threads 126 that mate with the first threads 118 of the first hub 108. The second end 124 of the second hub 110 can have the second threads 126 such as external threads on an external surface of the second hub 110. However, the second threads 126 can instead be internal threads on an internal surface of the second hub 110. Other configurations of the first threads 118 and the second threads 126 are also possible in order for the first threads 118 and the second threads 126 to mate with one another. The first threads 118 and the second threads 126 enable the first hub 108 and the second hub 110 to be screwed together to removably couple the first hub 108 and the second hub 110 together.

The proximal fitting 100 also includes a collet 128. The collet 128 can be a separate component from the first hub 108 and the second hub 110. The collet 128 has a passage 130 that extends from a first end 132 to a second end 134 of the collet 128. The first end 132 can be a proximal end and the second end 134 can be a distal end. The collet 128 can be configured to be inserted into the first end 114 of the passage 112 of the first hub 108. Furthermore, the first hub 108 and the second hub 110 can be configured to prevent the collet from passing through the second end 116 of the passage 112 of the first hub 108 and through the first end 122 of the second hub 110. For example, the collet 128 can be configured to be unable to pass through the second end 116 of the passage 112 of the first hub 108 and to be unable to pass through the first end 122 of the second hub 110. Therefore, the collet 128 can be restricted between the first hub 108 and the second hub 110 when the first hub 108 and the second hub 110 are coupled together.

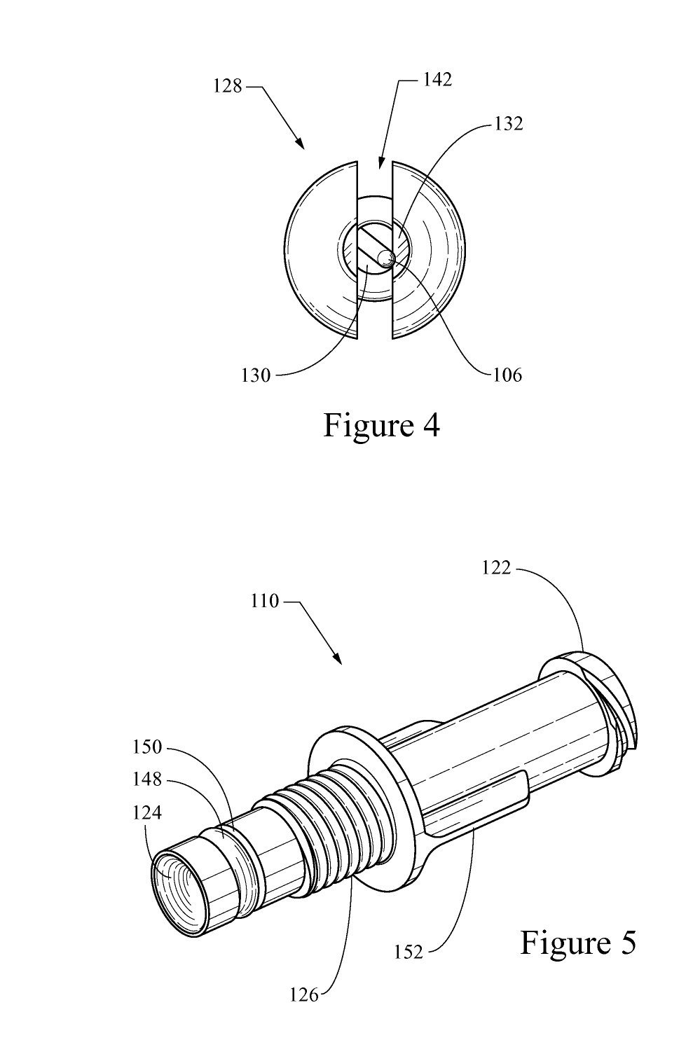

The collet 128 can include a first portion 136 that extends from the first end 132 to a position 138 between the first end 132 and the second end 134. The collet 128 can also include a second portion 140 that extends from the second end 134 to the position 138 between the first end 132 and the second end 134. The second portion 140 of the collet 128 can include at least one slot 142 that extends radially from the passage 130 in at least one direction. The cross-sectional view of FIG. 3 is a cross-section parallel to and through the slot 142 of the collet 128. FIG. 4 is a head on view of the first end 132 of the collet 128 with the guide wire extending through the passage 130. As shown in FIGS. 2-4, the slot 142 can extend across the first portion 136 to form two separate portions connected by the second portion 140 of the collet 128. The collet 128 may have a single slot 142 or may have more than one slot 142. For example, the collet 128 may have two slots that extend across the first portion 136 and cross at the passage 130 to form four separate portions connected by the second portion 140 of the collet 128.

The first portion 136 of the collet 128 can have a convex angular surface. The convex angular surface of the collet 128 can interface with a concave angular surface on the first hub 108 or the second hub 110. The concave angular surface is illustrated in FIGS. 1-3 as being on the second hub 110. For example, the concave angular surface can be on the second end 124 of the second hub 110. Alternatively, the convex angular surface of the collet 128 can interface with a concave angular surface on the first hub 108. The convex angular surface can interface with the concave angular surface thereby compressing or squeezing the passage 130 of the collet 128 in response to the first threads 118 and the second threads 126 engaging one another. The slot 142 allows the passage 130 to compress. For example, when the first portion 126 has two separate portions connected by the second portion 140, the two separate portions can deflect toward one another. Furthermore, even if the slot 142 does not extend entirely across the first portion 136, the passage 130 could still be configured to be compressible. The second portion 140 may not have a slot; therefore, passage 130 of the second portion 140 may not compress. The slot 142 may extend at least partially into the second portion 140 as well. When a guide wire 106 is within the passage 130 of the collet 128, the compression of the passage 130 can restrict movement of the guide wire 106. Furthermore, the force that the passage 130 applies to the guide wire 106 can be varied in relation to how far the first hub 108 and the second hub 110 are screwed together. For example, the first and second threads 118, 126 can have a sufficient thread pitch such as at least about 20 threads per inch to provide controllability of the amount of force that the collet 128 applies to the guide wire 106.

FIG. 5 is a perspective view of the second hub 110 illustrating the concave angular surface on the second end 124. The concave angular surface can be an inverted dome or have other configurations in which the surface is sloped or angled in a direction that generally extends inwardly from an outer surface of the second end 124. The convex angular surface of the collet 128 can be a dome or have other configurations in which the first end 132 of the collet 128 surface is beveled or curved. The concave angular surface and the convex angular surface can be curved, flat, or a combination thereof. The concave angular surface may, for example, have topology or contoured surface that is inverse or a mirror image to that of the topology or contoured surface of the convex angular surface. However, the concave and convex angular surfaces do not necessarily need to have corresponding contours.

The passage 112 of the first hub 108 can include a ledge 144 between the first end 114 and the second end 116. For example, the first end 114 of the passage 112 of the first hub 108 can have a cross-sectional area larger than a cross-sectional area of the second end 116 of the passage 112 of the first hub 108. The outer surface of the collet 128 can include a step 146 between the first portion 136 and the second portion 140 such that the step 146 interfaces with the ledge 144 to prevent the first portion 136 of the collet 128 from passing through the second end 116 of the first hub 108. For example, the first portion 136 can have a cross-sectional area larger than a cross-sectional area of the second portion 140. The second portion 140 of the collet 128 can also be configured to be inserted into at least a portion of the second end 116 of the first hub 108. The shape of second portion 140 of the collet 128 may correspond to the shape of the passage 112 at the second end 116 of the first hub 108 such that second portion 140 of the collet 128 can be close-fitting with the second end 116 of the passage 112 of the first hub 108. For example, the second portion 140 of the collet 128 can be tubular.

The proximal fitting 100 can further include an annular seal 148 disposed between the first hub 108 and the second hub 110 to seal the passage 130 of the collet 128 from the first threads 118 and the second threads 126. The annular seal 148 can be disposed in a groove in the first hub 108 and/or second hub 110. For example, as illustrated in FIGS. 3 and 4, the second hub 110 has a groove 150 disposed in an outer surface adjacent to the second end 124, and the annular seal 148 is disposed within the groove 150.

The passage 120 of the second hub 110 can have a cross-sectional area less than a cross-sectional area of the collet 128 such as a cross-sectional area of the first portion 136 so that the collet 128 cannot pass through the passage 120 of the second hub 110. However, the passage 120 of the second hub 110 can be tapered so that the guide wire 106 can be more easily inserted into the passage 120. For example, the passage 120 at the first end 122 of the second hub 110 can have a cross-sectional area larger than a cross-sectional area of the passage 120 at the second end 124 of the second hub 110.

As discussed above, the guide wire 106 can extend through the passages 112, 130, 120 of the first hub 108, the collet 128, and the second hub 110, respectively. The passages 112, 130, 120 can each be aligned with one another along an axis. When the passage 130 of the collet 128 is compressed, the collet 128 clamps onto the guide wire 106 and restricts movement of the guide wire 106. When the passage 130 of the collet 128 is compressed, the passage 112 of the first hub 108 can be in fluid communication with the passage 120 of the second hub 110. Furthermore, when the passage 130 of the collet 128 is not compressed, the passage 112 of the first hub 108 can be in fluid communication with the passage 120 of the second hub 110. For instance, a cross-sectional area of the guide wire 106 can be less than cross-sectional areas of the passages 112, 130, 120. As illustrated in FIG. 4, the guide wire 106 can have a diameter smaller than a diameter of the passage 130 of the collet 128. Therefore, fluid can pass around the guide wire 106 through the passages 112, 130, 120. Furthermore, even when the collet 128 compresses onto the guide wire 106, the slot 142 does not completely close because the guide wire 106 is sandwiched within the passaged 130. Therefore, fluid can pass through the slot 142 even when compressed. Having each of the passages 112, 130, 120 remain in fluid communication both when the collet 128 is compressed and uncompressed can enable the proximal fitting 100 to be flushed with a fluid. For example, the guide wire 106 may have a hydrophilic coating in which can be activated by flushing a fluid through the passages 112, 130, 120.

The second end 116 of the first hub 108 can be configured to be removably coupled to the catheter 102. Although a snap cap 104 is illustrated in FIGS. 1-3 to couple the first hub 108 to the catheter 102, other fasteners could be used. The second end 122 of the second hub 110 can be configured to be removably coupled to a syringe (not shown). For example, as illustrated in FIGS. 1-3 and 5, the second end 122 has a thread that can engage with a thread of a syringe. For instance, the second hub 110 can include a male luer lock adapter. Although FIGS. 1-3 show the first hub 108 coupled to the catheter 102, the proximal fitting 100 can still provide similar functionality if the second hub 110 is coupled to the catheter 102. For example, the second end 122 of the second hub 110 can be configured to be removably coupled to the catheter 102.

The proximal fitting 100 can advantageously be used as a torque device. When the collet 128 restricts movement of the guide wire 106, moving the proximal fitting 100 can move the guide wire 106. Therefore, the guide wire 106 can be rotated and moved laterally by rotating and moving laterally the proximal fitting 100. The first hub 108 and/or the second hub 110 can include finger grips 152. The finger grips 152 can help to apply torque to the proximal fitting 100 to move the guide wire 106. Furthermore, the finger grips 152 can help to apply torque to the first hub 108 and the second hub 110 to screw together and unscrew apart the first and second hubs 108, 110.

The proximal fitting 100 can be of a variety of dimensions depending on selected catheter and guide wire for a medical procedure. The passages 112, 130, 120 of the proximal fitting 100 can each have a cross-sectional area or diameter greater than a cross-sectional area or diameter of the guide wire 106 used with the proximal fitting 100. For example, the passages 112, 130, 120 can each have a cross-sectional area equal to or larger than the cross-sectional area of the catheter 102. The guide wire 106 may be a micro wire with a diameter less than about 18 thousandths of an inch. Furthermore, the passages 112, 130, 120 can each have a cross-sectional area or diameter greater than a cross-sectional area or diameter of a range of differently sized guide wires 106 so that the same proximal fitting 100 can be utilized in a variety of medical procedures.

The proximal fitting 100 can be formed from a variety of materials. For example, the first hub 108, the second hub 110, and the collet 128 can each be formed from a polymer such as nylon. Furthermore, the first hub 108, the second hub 110, and the collet 128 can each be manufactured or formed by machining, molding, etc. For example, the first hub 108, the second hub 110, and the collet 128 can each be formed by injection molding.

The proximal fitting 100 described herein can be used with various medical procedures. The guide wire 106 may be preloaded into the proximal fitting 100 prior to the medical procedure, or the guide wire 106 may be loaded during the medical procedure. The guide wire 106 can be positioned within the passage 112, 130, 120 so that the proximal end 154 of the guide wire 106 is within the passage 112, 130, 120. For example, the proximal end 154 of the guide wire 106 can be inside of the passage 120 of the second hub 110. The distal end of the guide wire 106 can extend out of the proximal fitting 100. For example, the distal end (not shown) of the guide wire 106 may extend out about 10 cm to about 20 cm from the proximal fitting 100. The catheter 102 can be attached to the proximal fitting 100 before or after the guide wire 106 has been inserted into the proximal fitting 100. The snap cap hub 104 can be used to couple a proximal end 156 of the catheter 102 to the second end 116 of the first hub 108. For example, as shown in FIG. 3, the snap cap 104 may have female luer threads to connect to the male luer threads 156 of the catheter 102. The second end 116 of the first hub 108 may also be pressed through the axial opening of the snap cap 104 to snap the groove of the first hub 108 into the snap cap 104 opening. The distal end of the guide wire 106 can extend out of the distal end of the catheter 102. The collet 128 of the proximal fitting 100 may be compressed onto the guide wire 106 prior to the medical procedure or during the medical procedure.

During a medical procedure, a syringe can be coupled to the first end 122 of the second hub 110 to flush the catheter and if the guide wire 106 has a hydrophilic coating, to activate the coating. With the collet 128 compressed onto the guide wire 106 and the catheter 102 attached to the proximal fitting 100, the distal end of the guide wire 106 can be inserted into an access site of the patient such as through an introducer so that the guide wire 106 can enter a vessel. The guide wire 106 and the catheter 102 can be moved along the vessel to a treatment sight in the vessel. After the catheter 102 is at the treatment sight, the proximal fitting 100 can be disconnected or separated from the catheter 102 by the snap cap 104 while the collet 128 remains compressed onto the guide wire 106. After the proximal fitting 100 is disconnected from the catheter 102, the proximal fitting 100 can be used as a torque device to rotate and move the guide wire 106 without rotating or moving the catheter 102. Adjustments of the distal end of the guide wire 106 can be made while keeping the proximal fitting 100 close to the proximal end 156 of the catheter. Such adjustments made to the distal end of the guide wire 106 may be needed to position the catheter 102 into a desired location. The guide wire 106 can then be removed from catheter 102.

Advantageously, the proximal fitting 100 can be removed from the guide wire 106 without separating the first hub 108 from the second hub 110. The first hub 108 can be only partially unscrewed relative to the second hub 110 so that the collet 128 releases the guide wire 106, but the first hub 108 and the second hub 110 remain at least partially coupled together. Therefore, when the catheter 102 is in the vessel, the guide wire 106 can be removed from the catheter 102 and the proximal fitting 100. A different guide wire can then be inserted into the proximal fitting 100 and the catheter 102. In another example, when the catheter 102 and the guide wire 106 are in the vessel, the proximal fitting 100 can be removed from the guide wire 106, and the catheter 102 can be removed from the vessel while the guide wire 106 remains in the vessel. A different catheter can then be slid over the guide wire 106 and inserted into the vessel. The proximal fitting 100 can be slid over the guide wire 106 after the different catheter has been inserted into the vessel.

The proximal fitting 100 can be used in other medical procedures as well. For example, the guide wire 106 can be inserted into a vessel without the proximal fitting 100. The catheter 102 can be slid over the guide wire 106 and inserted into the vessel. The proximal end 154 of the guide wire 106 can extend out the proximal end 156 of the catheter 102, and the proximal fitting 100 can be slid over the guide wire 106. The collet 128 of the proximal fitting 100 can be compressed onto the guide wire 106 and used as a torque device for the guide wire 106.

While preferred embodiments of the invention have been described, it should be understood that the invention is not so limited, and modifications may be made without departing from the invention. The scope of the invention is defined by the appended claims, and all devices that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

We claim:

1. A proximal fitting for a catheter comprising:
a first hub having a passage extending from a first end to a second end of the first hub, the first hub having first threads;
a second hub having a passage extending from a first end to a second end of the second hub, the second hub having second threads that mate with the first threads of the first hub, the first end of the first hub and the second end of the second hub being disposed toward each other, and the second end of the first hub and the first end of the second hub being disposed away from each other; and
a collet having a passage extending from a first end to a second end of the collet, the collet being configured to be inserted into the first end of the passage of the first hub while being unable to pass through the second end of the passage of the first hub and being unable to pass through the first end of the passage of the second hub, a first portion of the collet extends from the first end to a position between the first end and the second end, a second portion of the collet extends from the second end to the position, the first portion of the collet having a convex angular surface and at least one slot that extends radially from the passage in at least one direction, and the convex angular surface of the collet interfaces with a concave angular surface on the first hub or the second hub thereby compressing the passage of the collet in response to the first and second threads engaging one another.

2. The proximal fitting of claim 1, wherein the slot extends across the first portion to form two separated portions connected by the second portion.

3. The proximal fitting of claim 1, wherein the second portion of the collet is tubular.

4. The proximal fitting of claim 1, wherein the slot extends partially into the second portion of the collet.

5. The proximal fitting of claim 1, wherein the passage of the first hub comprises a ledge between the first end and the second end, and an outer surface of the collet comprises a step between the first portion and the second portion such that the step interfaces with the ledge to prevent the first portion of the collet from passing through the second end of the first hub.

6. The proximal fitting of claim 1, wherein the second end of the collet is configured to be inserted into at least a portion of the second end of the first hub.

7. The proximal fitting of claim 1, wherein the passage at the first end of the second hub has a cross-sectional area larger than a cross-sectional area of the passage at the second end of the second hub.

8. The proximal fitting of claim 1, further comprising an annular seal disposed between the first and second hubs to seal the passage of the collet from the first and second threads.

9. The proximal fitting of claim 1, further comprising finger grips on at least one of the first hub or the second hub.

10. The proximal fitting of claim 1, wherein the concave surface on the first hub or the second hub is on the second end of the second hub.

11. The proximal fitting of claim 1, wherein the first threads are internal threads and the second threads are external threads.

12. The proximal fitting of claim 1, in combination with a guide wire extending through the passages of the first hub, the collet, and the second hub.

13. The proximal fitting of claim 12, wherein when the passage of the collet is compressed, the collet clamps onto the guide wire and restricts movement of the guide wire.

14. The proximal fitting of claim 12, wherein when the passage of the collet is compressed, the passage of the first hub is in fluid communication through the slot with the passage of the second hub.

15. The proximal fitting of claim 12, wherein a cross-sectional area of the guide wire is less than cross-sectional areas of the passages of the first hub, the collet and the second hub.

16. The proximal fitting of claim 12, wherein the guide wire has a hydrophilic coating.

17. The proximal fitting of claim 1, wherein the second end of the first hub is configured to be removably coupled to a catheter.

18. The proximal fitting of claim 1, wherein the first end of the second hub is configured to be removably coupled to a syringe.

19. The proximal fitting of claim 1, further comprising an annular seal disposed between the first and second hubs to seal the passage of the collet from the first and second threads, wherein the second end of the first hub is configured to be removably coupled to a catheter, the first end of the second hub is configured to be removably coupled to a syringe.

20. The proximal fitting of claim 1, further comprising an annular seal disposed between the first and second hubs to seal the passage of the collet from the first and second threads, and wherein the slot extends across the first portion to form two separated portions connected by the second portion; and
in combination with a guide wire extending through the passages of the first hub, the collet, and the second hub, wherein a cross-sectional area of the guide wire is less than cross-sectional areas of the passages of the first hub, the collet and the second hub, and further wherein when the passage of the collet is compressed, the collet clamps onto the guide wire and restricts movement of the guide wire and the passage of the first hub is in fluid communication with the passage of the second hub.

21. The proximal fitting of claim 20, wherein the concave surface on the first hub or the second hub is on the second end of the second hub, the first threads are internal threads and the second threads are external threads, the second end of the first hub is configured to be removably coupled to a catheter, and the first end of the second hub is configured to be removably coupled to a syringe
